(19) United States
(12) Patent Application Publication
(10) Pub. No.: US 2025/0090578 A1
(43) Pub. Date: Mar. 20, 2025

(54) METHOD OF PRODUCING A POPULATION OF IMMUNE CELLS FROM PLURIPOTENT STEM CELLS

(71) Applicant: REPAIRON IMMUNO GMBH, Goettingen (DE)

(72) Inventors: Mine BAKAR, Goettingen (DE); Lothar GERMEROTH, Goettingen (DE)

(73) Assignee: REPAIRON IMMUNO GMBH, Goettingen (DE)

(21) Appl. No.: 18/290,710

(22) PCT Filed: Jul. 19, 2022
(86) PCT No.: PCT/EP2022/070224

§ 371 Date: Jan. 19, 2024

(30) Foreign Application Priority Data
Jul. 19, 2021 (EP) 21186400.4

(51) Int. Cl. A61K 35/17; A61K 39/00; C12N 5/0783

(52) U.S. Cl. CPC A61K 35/17; A61K 40/11; A61K 40/42; C12N 5/0636; C12N 5/0646; C12N 2500/38; C12N 2501/115; C12N 2501/125; C12N 2501/145; C12N 2501/155; C12N 2501/16; C12N 2501/165; C12N 2501/2302; C12N 2501/2307; C12N 2501/2315; C12N 2501/26; C12N 2501/42; C12N 2501/515; C12N 2501/727; C12N 2506/45; C12N 2513/00

(57) ABSTRACT

The present invention relates to a method of producing a population of immune cells, the population of immune cells obtainable or obtained by the method of the invention, non-immunogenic T cell obtainable or obtained by the method of the invention, pharmaceutical composition comprising the immune cells or non-immunogenic T cells of the invention, as well as to the population of immune cells, the non-immunogenic T cell or the pharmaceutical composition for use in a method of treatment, or the use in cell therapy.

### TECHNICAL FIELD OF THE INVENTION

The present invention relates to a method of producing a population of immune cells, the population of immune cells obtainable or obtained by the method of the invention, non-immunogenic T cell obtainable or obtained by the method of the invention, pharmaceutical composition comprising the immune cells or non-immunogenic T cells of the invention, as well as to the population of immune cells, the non-immunogenic T cell or the pharmaceutical composition for use in a method of treatment, or the use in cell therapy.

### BACKGROUND OF THE INVENTION

The development of immune cell populations, in particular T cell populations, for use in diagnosis and therapy is a current task. To be able to provide such immune cell populations, such as differentiated T cell populations, stem cells are a good source. The crucial characteristic of stem cells is their capability to self-renew indefinitely and to differentiate in multiple different types of cells or tissue. The self-renewal capability of stem cells is crucial for their characteristic as pool of primitive undifferentiated cells. Further, the characteristic of high “flexibility” and “plasticity” of stem cells is founded on their capability to trans-differentiate into tissues which may be different from their origin. Pluripotent stem cells (PSC) are in the focus as source for the generation of T cells. In particular induced pluripotent stem cells (iPS), which represent non-embryonic cells that are reprogrammed to pluripotent stem cells, are a useful source. Currently, the most utilized pluripotent cells are embryonic stem cells (ESC) or induced pluripotent stem cells (iPSC). Human ESC-lines were first established by Thomson and coworkers (Thomson et al. (1998), Science 282:1145-1147). Human ESC research recently enabled the development of a new technology to reprogram cells of the body into an ES-like cell. This technology was pioneered by Yamanaka and coworkers in 2006 (Takahashi & Yamanaka (2006), Cell, 126:663-676). Resulting induced pluripotent cells (iPSC) show a very similar behavior as ESC and, importantly, are also able to differentiate into every cell of the body. Moreover, it was reported that also parthenogenetic stem cells are suitable for EHM-production in a mouse model (Didié et al. (2013), J Clin Invest., 123:1285-1298). The generation of hypoimmunogenic T cells from genetically engineered allogeneic human induced pluripotent stem cells is described, in Wang et al., Nature Biomedical Engineering, Vol. 5, May 2021, 429-440, for example. T cells, which are individually engineered, are a powerful tool for the use in targeted therapies of diseases which require a directed immune response, such as cancer. It has been shown that T cells can be cultured and proliferated in vitro for use in adoptive cellular immunotherapy or cancer therapy in which such T cells have been proofed to possess anti-tumor activity in a patient having a tumor.

Methods for the production of T cells derived from PSC are known in the art, see Wang et al, 2021, supra. In addition, Montel-Hagen et al. (Montel-Hagen et al., 2019, Cell Stem Cell 24, 376-389, Mar. 7, 2019) disclose a method for the production of T cells from pluripotent stem cells. According to this publication, immune cell differentiation is achieved by culturing the cell aggregates in an air-liquid interface setting. Thus, the method as described in Montel-Hagen includes a complicated and time-consuming cell culture technique for the production of T cells from PSCs. Further, Iriguchi et al. (Iriguchi et al., 2021, Nature Communications, 12:430 January 18; 12 (1): 430. doi: 10.1038/s41467-020-20658-3) disclose a method for the production of T cells from iPSC. Iriguchi et al. describe a method which foresees T cell differentiation on DLL4 coated plates with the addition of further factors. This requires some cell culture capacity, making it difficult to produce higher numbers of differentiated T cells. These methods have the drawback that these methods are rather laboursome and not suitable to produce immune cells in a cost-effective way. Moreover, these prior art methods allow only the production of T cells.

Therefore, there is a need for the provision of a method which allows the production of a population of immune cells, such as T cell like cells but ideally also NK cell like cells., This method should also be suitable to be conducted in a large-scale approach, for example, in a bioreactor, to achieve larger yields of the desired immune cells in an efficient way.

Consequently, it is an object of the present invention to provide such a method for producing a population of immune cells which fulfils said demand. This object is solved by the subject-matter of the independent claims, in particular by the method for producing a population of immune cells, the population of immune cells obtainable or obtained by said method, the T cell obtainable or obtained by said method, the pharmaceutical composition and the population of immune cells for use in a method of treatment. The provision of a population of immune cells as described here has the added advantage that it is possible to selectively produce a distinct subpopulation of the population of immune cells, in particular T cell like cells and/or NK cell like cells, dependent on the desired application.

### SUMMARY OF THE INVENTION

A first aspect of the invention relates to a method of producing a population of immune cells from pluripotent stem cells (PSC), the method comprising the steps of: (i) inducing 3D cell aggregate formation and hematopoietic differentiation by (a) culturing under suitable conditions PSC having undergone mesenchymal differentiation in the presence of Notch-ligand Delta-like ligand 4 (DLL4) signaling activity, on a solid support suitable for formation of 3D-cell aggregates, in a serum-free medium, thereby allowing the formation of 3D-cell aggregates; (b) collecting the 3D-cell aggregates and culturing under suitable conditions in a suspension culture in a serum-free medium for about 3 to 6 days; and (c) culturing the 3D-cell aggregates under suitable conditions for about further 4 to 7 days in a serum-free medium; (ii) inducing immune cell differentiation by culturing the 3D-cell aggregates of (i) under suitable conditions in a suspension culture for a suitable time in a serum-free medium; thereby providing a population of immune cells.

A second aspect of the invention relates to a population of immune cells obtainable by the method of the invention.

A third aspect of the invention relates to a population of immune cells obtained by the method of the invention.

A fourth aspect of the invention relates to a non-immunogenic T cell obtainable by the method of the invention.

A fifth aspect of the invention relates to a non-immunogenic T cell obtained by the method of the invention.

A sixth aspect of the invention relates to a non-immunogenic T cell obtainable by the method of the invention, wherein the T cell is deficient of endogenous MHC class I molecules presented on the cell surface of the T cell and the T cell comprises an immunomodulatory protein on its surface.

A seventh aspect of the invention relates to a pharmaceutical composition comprising the population of immune cells of the invention, or the non-immunogenic T cell of the invention.

An eighth aspect of the invention relates to the population of immune cells of the invention, the non-immunogenic T cell of the invention, or the pharmaceutical composition of the invention for use in a method of treatment.

Finally, a ninth aspect of the invention relates to the population of immune cells of the invention, the non-immunogenic T cell of the invention, or the pharmaceutical composition of the invention for use in cell therapy.

### BRIEF DESCRIPTION OF THE FIGURES

FIG. 1 shows a schematic overview of the time line of an illustrative embodiment of the method of the present invention.

FIG. 2 shows microscopic images of cells seeded into AggreWell™800 6-well plate (FIG. 2A) on day 0, (FIG. 2B) on day 3 before being differentiated into a population of immune cells by a method as described herein.

FIG. 3 shows a flow cytometric analysis of a population of immune cells including T cell like cells and NK cell like cells produced by a method of the present invention. Cell progenitors were pre-gated on single, live, lymphocyte-like cell population according to side and forward scatter. To separate progenitors from feeders, cells were gated according to CD29/CD45 features. All CD29⁻CD45⁺ cells were characterized for phenotype of CD3⁺CD56⁻ (T cell lineage marker), CD3⁻CD56⁺CD8^{+/-} (NK cell lineage marker) and CD19⁺ (B cell lineage marker). Moreover, CD3⁺CD56⁻ T cell-like cells were further characterized for expression of CD4 and CD8 markers. These cells exhibited expression pattern similar to classical thymocyte profile of CD4⁻CD8⁻, CD4⁺CD8⁺, CD4⁻CD8⁺ and CD4⁺CD8⁻.

FIG. 4 shows a flow cytometric analysis of cells of the population of immune cells as obtained herein after stimulation with an anti-human CD3 antibody. Cell progenitors were pre-gated on single, live, lymphocyte-like cell population according to side and forward scatter. To separate progenitors from feeders, cells were gated according to CD29/CD45 features. All CD29⁻CD45⁺ cells were characterized for phenotype of CD3⁺CD56⁻ (T cell lineage marker), or CD3⁻CD56⁺ (NK cell lineage marker).

FIG. 5 shows two diagrams. The diagram above shows the cell counts of absolute CD45+CD56+ NK like cells having undergone the (differentiation) method of the present invention and being further expanded on day 0 (start of the expansion) and on day 13 of the expansion. The diagram below shows that the CD45+CD56+ NK like cells have 19-fold expanded from day 0 to day 13.

FIG. 6 shows the result of the lactate dehydrogenase (LDH) activity assay of the differentiated immune cells. The diagram in the upper part of FIG. 6 represents the means +/−SD of triplicate cultures of K562 target cells and differentiated immune cells of the invention. In the lower part of FIG. 6 a microscopic analysis is shown of the control and coculture groups of K562 cells and differentiated immune cells of the invention as used in the LDH assay.

### DETAILED DESCRIPTION OF THE INVENTION

A first aspect of the invention is directed to a method of producing a population of immune cells from pluripotent stem cells (PSC), the method comprising the steps of: (i) inducing 3D cell aggregate formation and hematopoietic differentiation by (a) culturing under suitable conditions PSC having undergone mesenchymal differentiation in the presence of Notch-ligand Delta-like ligand 4 (DLL4) signaling activity, on a solid support suitable for formation of 3D-cell aggregates, in a serum-free medium, thereby allowing the formation of 3D-cell aggregates; (b) collecting the 3D-cell aggregates and culturing under suitable conditions in a suspension culture in a serum-free medium for about 3 to 6 days; and (c) culturing the 3D-cell aggregates under suitable conditions for about further 4 to 7 days in a serum-free medium; (ii) inducing immune cell differentiation by culturing the 3D-cell aggregates of (i) under suitable conditions in a suspension culture for a suitable time in a serum-free medium; thereby providing a population of immune cells. In this context it is noted that it has been surprisingly found in the present invention that such a method does not only provide T-cells as described by Montel-Hagen et al., 2019, supra or Iriguchi et al., 2021, supra but allows to produce a distinct subpopulation of the population of immune cells, in particular T cell like cells and/or NK cell like cells, simply dependent on the intended use of the resulting immune cells.

As used herein, the term “about” when used with reference to intervals or periods of time has to be understood such that also any interval which deviates from the distinct value of the given interval is also comprised. For example, an interval of about 3 to 6 days, which means 72 to 144 hours, also comprises intervals which deviate for 1, 2, 3, 4, 5 or 6 hours from the given interval, such that the given interval may be 1, 2, 3, 4, 5, or 6 hours shorter or longer concerning the beginning and/or the end to the interval.

One advantage of the present invention is that it allows using a so-called feeder-free system. This means that inducing 3D-cell aggregates and hematopoietic differentiation can be carried out without the use/help of stromal cells. Thus, in a preferred embodiment the Notch-ligand Delta-like ligand 4 (DLL4) signaling activity is not provided by a cell, but instead by alternative means/structures. Such an alternative structure may be, for example, be a bead coated with DLL4, as described in more detail below. It may also be possible to use soluble versions of DLL4 to provide the DLL4 signaling activity. In view of the subsequent steps of the method of the present invention, such feeder-free approach provides the advantage that possibly contaminating cells can be avoided and do not have to be deleted or sorted out when conducting the method of the invention. Therefore, the feeder-free system of the present invention represents a pure system which is also efficient since additional purification steps are made obsolete.

In a further preferred embodiment of the invention, the method includes that before step (i) a further step (0i) is conducted, which comprises: (0i) inducing mesodermal differentiation by (a) seeding PSC under suitable conditions in a serum-free medium; and (b) culturing the PSC under suitable conditions in a serum-free medium for about 2 to about 4 days, and (c) resuspending cells with serum-free medium. Preferably, the method of the present invention does not require any additional substances or treatment before the mesodermal differentiation.

In one embodiment of the method of the invention the serum-free medium may preferably: in step (0i) (a) Activin A, bone morphogenic protein 4 (BMP4), vascular endothelial growth factor (VEGF), fibroblast growth factor (FGF), and an apoptosis inhibitor, preferably a ROCK inhibitor, each in a concentration of about 10 to about 50 ng/mL; (b) BMP4, VEGF, and FGF, each in a concentration of about 5 to 15 ng/ml; (c) an apoptosis inhibitor, preferably a ROCK inhibitor; in step (i) (a) an inhibitor of ALK receptors and a ROCK inhibitor, each in a concentration of about 5 to about 15 μM, (b) about 5 to about 15 ng/mL of an inhibitor of ALK receptors, (c) about 5 to about 15 ng/ml of an inhibitor of ALK receptors, about 40 to about 60 ng/ml stem cell factor (SCF), about 5 to about 15 ng/ml FMS-like tyrosine kinase 3 ligand (Flt-3l), and about 5 to about 15 ng/mL Thrombopoietin (TPO); in step (ii) about 1% to about 3% B27 supplement, about 5 to about 15 ng/ml SCF, about 5 to about 15 ng/mL Flt-3l, about 5 to about 15 ng/ml IL-7, and about 15 to about 45 mM L-ascorbic acid 2-phophatate sesquimagnesium salt hydrate.

As used herein, the term “about” in relation to any value of a concentration or range of a concentration has to be understood such that any concentration or range of concentration which deviates from the distinct value of the given concentration or range of concentration is also comprised. For example, a concentration of about 50 ng/ml comprises also a lower concentration of 40, 41, 42, 43, 44, 45, 46, 47, 48, or 49 ng/ml, or a higher concentration of 51, 52, 53, 54, 55, 56, 57, 58, or 59 ng/mL.

In a preferred embodiment of the invention, the ROCK inhibitor may be Y-27632 dihydrochloride and the inhibitor of ALK receptors may be SB43152. The use of the ROCK inhibitor provides the advantage that apoptosis and de-differentiation is limited/avoided. Accordingly, the use of ROCK inhibitor and of inhibitor of ALK receptors avoids the loss of cells due to apoptosis which means that a distinct quantity of cells is provided with the method of the invention. In addition, the loss of differentiation is reduced since the cells maintain the desired differentiation characteristics. This means that a distinct quality of cells is provided using the method of the present invention.

Preferably, the suspension culture in step (i) and (ii) is a dynamic suspension culture, that means a suspension culture that is being agitated, for example by culturing on a shaking device. Any suitable shaking device/shaker can be used, for example an orbital shaker, a horizontal shaker or a linear shaker. The culture can be moved/agitated under any suitable conditions that can be experimentally determined by the skilled person. For example, a shaker such an orbital shaker may be operated at a rate of about 60 to about 80 rotations per minutes (rpm). In contrast to an adherent cell culture, the suspension cell culture as used herein provides several advantages. Adherent cell culture requires more space and requires a more elaborate handling, for example for passaging of cells. The suspension culture is a cell culture format which allows to provide a scalable cell culturing process which is useful for the provision of higher cell numbers. Such higher cell numbers are in particular required for clinical use of cellular products. The dynamic suspension culture can also be much easier transferred to bioreactor conditions compared to an adherent cell culture, such as 2D cell culture or static cell culture, since the features of the dynamic cell culture are already very similar to the features of a culture in a bioreactor. Moreover, growth factors which are present in the culture medium are able to circulate in the dynamic cell culture. This allows that every cell can be in close contact with such growth factors. This may positively influence the culture process, in particular the differentiation process of the cells and can help to achieve a more homogenous and better-defined cell population.

The induction of the immune cell differentiation can be carried out over any suitable time. A suitable time can be determined experimentally, for example, by taking cell samples over a certain period of time and determine the nature/differentiation state of the cells, for example by analysis of the cell surface markers (see the experimental section where cells were first screened for CD29/CD45 markers and all CD29−CD45+ cells were characterized for phenotype of CD3+CD56− (T cell lineage marker), CD3−CD56+CD8+/− (NK cell lineage) and CD19+ (B cell lineage). By so doing the suitable time for differentiation can be determined, for example, also depending on which cell type, for example, a NK cell like immune cell or a T cell like immune cell is wished to be obtained as product of the method of the invention. In a preferred embodiment of the invention, the step (ii) of inducing immune cell differentiation by culturing 3D—cell aggregates of (i) under suitable conditions in a suspension culture is carried out conducted for about 21 to about 50 days in serum-free medium.

The term “pluripotent stem cell” (PSC) as used herein refers to any such stem cell type that is able to differentiate into a population of immune cells. In the context of the present invention, these pluripotent stem cells are preferably not produced using a process which involves modifying the germ line genetic identity of human beings or which involves use of a human embryo for industrial or commercial purposes. Preferably, the pluripotent stem cells are of primate origin, more preferably human. In a further preferred embodiment of the invention, i the PSC are human induced pluripotent stem (iPS) cells, preferably the human iPS cell line TC-1133. The cell line TC-1133 have been reprogrammed under c-GMP conditions and can be purchased from Lonza. Further suitable PSC's, including induced PSCs, can for example, be obtained from the NIH human embryonic stem cell registry, the European Bank of Induced Pluripotent Stem Cells (EBISC), the Stem Cell Repository of the German Center for Cardiovascular Research (DZHK), or ATCC, to name only a few sources. Pluripotent stem cells are also available for commercial use, for example, from the NINDS Human Sequence and Cell Repository (https://stemcells.nindsgenetics.org) which is operated by the U.S. National Institute of Neurological Disorders and Stroke (NINDS) and distributes human cell resources broadly to academic and industry researchers. Human iPS cells may have cultured on Matrigel-coated container, such as Matrigel-coated T-flasks. The iPS cells are preferably cultured in iPS expansion medium, such as Miltenyi StemMACS iPS-Brew XF. Further exemplary iPSC cell lines that can be used in the present invention, include but are not limited to, the Human Episomal iPSC Line of Gibco™ (order number A18945,Thermo Fisher Scientific), or the iPSC cell lines ATCC ACS-1004, ATCC ACS-1021, ATCC ACS-1025, ATCC ACS-1027 or ATCC ACS-1030 available from ATTC. Alternatively, any person skilled in the art of reprogramming can easily generate suitable iPSC lines by known protocols such as the one described by Okita et al, “A more efficient method to generate integration-free human iPS cells” Nature Methods, Vol.8 No.5, May 2011, pages 409-411 or by Lu et al “A defined xeno-free and feeder-free culture system for the derivation, expansion and direct differentiation of transgene-free patient-specific induced pluripotent stem cells”, Biomaterials 35 (2014) 2816e2826. The (induced) pluripotent stem cell that is used in the present invention can be derived from any suitable cell type (for example, from a stem cell such as a mesenchymal stem cell, or an epithelial stem cell or a differentiated cells such as fibroblasts) and from any suitable source (bodily fluid or tissue). Examples of such sources (body fluids or tissue) include cord blood, skin, gingiva, urine, blood, bone marrow, any compartment of the umbilical cord (for example, the amniotic membrane of umbilical cord or Wharton's jelly), the cord-placenta junction, placenta or adipose tissue, to name only a few. In one illustrative example, is the isolation of CD34-positive cells from umbilical cord blood for example by magnetic cell sorting using antibodies specifically directed against CD34 followed by reprogramming as described in Chou et al. (2011), Cell Research, 21:518-529. Baghbaderani et al. (2015), Stem Cell Reports, 5(4): 647-659 show that the process of iPSC generation can be in compliance with the regulations of good manufacturing practice to generate cell line ND50039. Accordingly, the pluripotent stem cell preferably fulfils the requirements of the good manufacturing practice.

The PSC used in the present invention may be pluripotent stem cells that are deficient of endogenous MHC class I molecules presented on the cell surface of the pluripotent stem cell and that comprise an immunomodulatory protein on their surface.

A cell or pluripotent stem cell, which is “deficient of endogenous MHC class I molecules presented on the cell surface” does not present a functional MHC class I molecule on its surface, i.e. the surface of the cell or the pluripotent stem cell. Nor does such a deficient cell/pluripotent cell comprise a functional MHC class I molecule in its cell membrane. In this context, the term “endogenous” relates to any MHC class protein I, which naturally is comprised in the cell or pluripotent stem cell and not artificially introduced. Having endogenous MHC class I molecules increases the risk of a rejection reaction of the immune system of the recipient because a lack of MHC class I molecules on the cell surface might be interpreted as a “missing self”-signal by the immune system. Accordingly, the feature that the cell or pluripotent stem cells is deficient of MHC class I molecules on their surface, does not apply to any immunomodulatory protein, which may be introduced into the pluripotent stem cell and/or a recombinant immunomodulatory protein. In one embodiment, the deficiency of MHC class I molecules on the cell surface can be achieved by disrupting all copies of the beta 2-microglobulin gene in the pluripotent stem cells. The MHC complex is a heterodimer of alpha-microglobulin and beta 2-microglobulin. Hence, if beta 2-microglobulin is missing, no functional MHC class I complex can be assembled and consequently, no MHC class I molecule is present on the cell membrane and/or cell surface. Many possible ways are known to the person skilled in the art to modify the genome of the pluripotent stem cell in such a way that they are deficient of MHC class I molecules and comprise an immunomodulatory protein. It should be noted that a pluripotent stem cell of the invention, which is deficient of MHC class I molecules, may express an immunomodulatory protein, even if it is an MHC class I molecule such as HLA-E described herein. Accordingly, the term “deficient of MHC class I molecules” may relate to endogenous MHC class I molecules and does not exclude the presence of a (recombinant) immunomodulatory protein.

In a further preferred embodiment of the invention the immunomodulatory protein is a single chain fusion HLA class I protein.

Preferably, the single chain fusion HLA class I protein may comprise at least a portion of B2M covalently linked to at least a portion of an HLA class Iα chain selected from the group consisting of HLA-A, HLA-B, HLA-C, HLA-E, HLA-F and HLA-G.

In a preferred embodiment of the invention the pluripotent stem cell expresses further a target peptide antigen that is presented by the single chain fusion HLA class I protein on the pluripotent cell surface.

Preferably, the target peptide antigen is covalently linked to the single chain fusion HLA class I protein.

In a preferred embodiment the target peptide antigen comprises the sequence VMAPRTLFL (SEQ ID NO: 1).

Preferably, essentially all copies of the beta-microglobulin 2 gene are disrupted in the pluripotent stem cells. In the pluripotent stem cells of the invention the B2M gene may be disrupted so that no functional endogenous B2M protein is produced from the disrupted genetic loci. In certain embodiments, the disruption results in expression of non-functional B2M proteins, including but not limited to truncations, deletions, point mutations and insertions. In other embodiments, the disruption results in no protein expression from the B2M gene.

In a preferred embodiment the pluripotent stem cells are selected from the group consisting of embryonic stem cells, induced pluripotent stem cells and parthenogenetic stem cells.

Preferably, the pluripotent stem cells are pluripotent stem cells of primate origin, preferably human pluripotent stem cells.

In a preferred embodiment the pluripotent stem cell generated from CD34-positive cell are isolated form umbilical cord blood.

Preferably, the pluripotent stem cell used in the present invention is ND-50039 of the NINDS Human Cell and Data Repository.

In a preferred embodiment the PSC pluripotent stem cells are being deficient of endogenous MHC class II.

Preferably, the PSC are deficient of endogenous MHC class II by disrupting the C2TA gene-MHC class II transactivator.

In a preferred embodiment of the method of the present invention in step (i) Notch-ligand Delta-like ligand 4 (DLL4) signaling activity is provided by co-culturing PSC with stromal cells expressing DLL4, or alternatively by incubating PSC with beads which are coated with DLL4.

Incubating PSC with beads which are coated with DLL4 provides the advantage that the DLL4 signaling activity is provided without the need of any additional cells, such as stromal cells. Accordingly, a so-called feeder-free system can be provided with the method of the present invention. Thus, this avoids any steps of sorting or selecting DLL4-expressing cells out of the population of immune cells provided by the method of the invention. If necessary, DLL4-coated beads may be separated by standard methods know to person skilled in the art, such as magnetic separation procedures or flow cytometric procedures. A person skilled in the art knows different kind of beads which are suitable to be used for DLL4 coating, such as microbeads or beads with a larger diameter, for example dynabeads (Thermo Fisher Scientific).

When stromal cells are used, the ratio of PSC to stromal cells may be in the range of about 1:5 to about 1:40. Such a range is advantageous, since it allows to use a lower number of stromal cells compared to the method of the prior art, such as Montel-Hagen et al., 2019, supra. Therefore, in the case lower number of stromal cells are being used, this may have a beneficial influence on the composition of growth factors and the concentration of growth factors secreted by the stromal cells. For example, a lower number of stromal cells will likely produce decreased amounts of growth factor(s) resulting in a reduced concentration of growth factor(s) in the medium. It is assumed that such a reduced concentration of growth factors will have an advantageous influence on the differentiation of the pluripotent stem cells. Further, a lower number of stromal cells requires a lower effort to delete these stromal cells later in the procedure.

In a preferred embodiment of the invention that makes of stromal cells, the stromal cells used in in step (i) are the bone marrow-derived mouse stromal cell line MS5. Mouse stromal cells are advantageous since these cells are able to support immune cell differentiation.

In a further preferred embodiment, in step (i) the stromal cells are stromal cells differentiated from PSC expressing DLL4. The use of such stromal cells provides the advantage that the method of the present invention does not require any mouse derived cell which may have to be deleted or sorted out later in the procedure. Therefore, it is possible to provide a population of immune cells which does not include any non-human cells, such as mouse cells.

In a preferred embodiment the PSC are cultured during induction of mesodermal differentiation in step (0i) in a cell culture medium/container that comprises at least one extracellular matrix protein. Preferably, about 0.5 to about 2.0×10⁶cells are seeded in the respective cell culture container, such as a well of a 6-well plate. Adjusting the cell number of the PSC within this distinct range may have impact the growth factor ratio in the medium and may also influence the subsequent differentiation. The ranges of cell numbers mentioned above allows the beneficial differentiation of cells into the desired population of immune cells (see the experimental section).

Preferably, the container is coated with the at least one extracellular matrix protein.

In a preferred embodiment the at least one extracellular matrix protein may be selected from the group consisting of vitronectin, laminin, collagen, fibronectin, elastin, Matrigel, a peptide containing the amino acid sequence RGD, a protein containing the amino acid sequence RGD and combinations thereof. Accordingly, to provide an optimal structural and biochemical environment for the PSC and the subsequent differentiation procedure. The skilled person is able to empirically select the respective optimal extracellular matrix protein or the optimal combination of extracellular matrix proteins. Thus, also a combination of two, three, four or more extracellular matrix proteins can be used in the method of the present invention.

Preferably, in step (i)(a) of the present method the solid support comprises one or more wells, wherein each well comprises a V-shaped or conical cavity.

In a preferred embodiment the solid support is a microwell culture plate. Any suitable microwell culture plate can be used as long as it allows the generation of the desired immune cell population. Examples of suitable microwell culture plates include, but are not limited to, an AggreWell™ plate (available from StemCell Technologies), an BIOFLOAT™ 96-well plate (available from faCellitate), or an Nunclon Sphera 3D culture (available from ThermoFisher). Such cell culture plates are advantageous as they allow the beneficial dynamic suspension cell culture as described here. In case an AggreWell™ plate is, for example, used, this plate is preferably pre-treated with an anti-adherence solution. Such an anti-adherence solution allows to reduce surface tension and prevents cell adhesion. This provides a low adherence surface which facilitates the formation of 3D-cell aggregates. The formation of 3D-cell aggregates can be conducted in a very easy and robust way which allows also scale-up. The 3D-cell aggregate formation is different compared with prior art methods such as described in Montel-Hagen et al., 2019 supra which describe complicated and time-consuming cell culture techniques that are not suitable for scale-up. In addition, the 3D-cell aggregates as obtained using the method of Montel-Hagen et al., 2019 supra appear to be larger compared to the 3D-cell aggregates of the method of the present invention. This provides the added advantage of the present invention that growth factors can more easily diffuse into such smaller 3D-cell aggregates compared to larger 3D-cell aggregates. Thus, cells in the inner place of the 3D-cell aggregate can be more efficiently reached by the growth factors leading to efficient and more homogenous differentiation of all cells of the 3D-cell aggregate. See Example 1 of the present application in this respect.

In a preferred embodiment the 3D-cell aggregates after step (ii) are separated such that single cells are provided.

As mentioned above, it has been surprisingly found that in method of the present invention the population of immune cells may comprise both T cell like cells and NK cell like cells, that means a mixture of different cell types.

Thus, the term “population of immune cells” as used herein includes a mixture of different immune cells which is comprised in the population of immune cells. Thus, “a population of immune cells” should not be understood as a single homogenous group of immune cells such as a pure or homogenous population of T cells (wherein homogeneity can be assessed by the percentage of cells that express or lack expression of specific markers proteins. The term “population of immune cells” means different immune cells or different types of immune cells, which can be distinguished from each other and subsequently also isolated from each other by a skilled person with methods known in the art, such as flow cytometric methods. Thus, the isolation and subsequent expansion of the specific and homogenous cell population that is characterised by the expression (pattern) of specific marker proteins is indeed part of the present invention (cf. Example 4 of the present application in which after differentiation CD45+ CD56+ NK cell like cells were expanded). The term “population of immune cells” means that any type of immune cell may be comprised in the population of cells obtained by the present method. Immune cells comprise neutrophils, eosinophils (acidophiles), basophils, lymphocytes, and monocytes, and among the lymphocytes B cells, T cells and natural killer (NK) cells. Further, the population of immune cells according to the invention can be considered as a population of immune cells that have any (wanted) differentiation level or any intermediate differentiation level of an immune cell. The population of immune cells may also comprise cells representing precursor cells of T cells or T cell like cells or precursor cells of NK cells or NK cell like cells as well as cells representing mature T cells or T cell like cells or mature NK cells or NK cell like cells. Particularly, the population of immune cells according to the present invention comprises immune cells which have a phenotype of T cell like cells and of NK cell like cells.

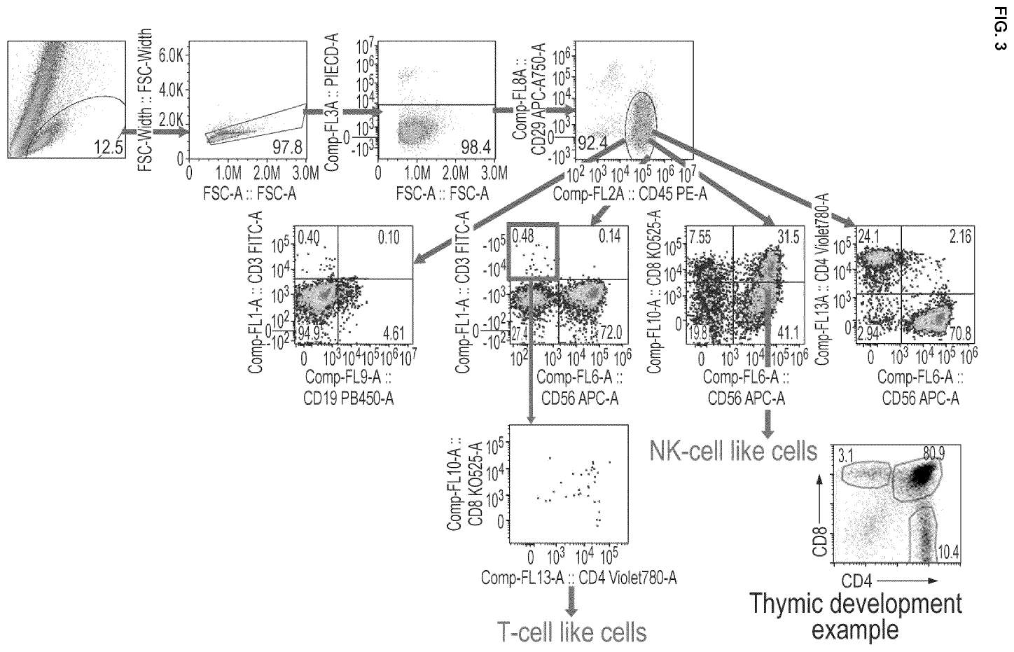

The provision of a population of immune cells has the advantage that it is possible to select between the resulting T cell like cells and NK cell like cells. Therefore, in contrast to the methods of the prior art which can only provide T cells or T cell like cells, the method of the present invention is able to provide T cell like cells together with NK cell like cells. Therefore, dependent on the desired application a choice can be made between T cell like cells or NK cell like cells. The yield of these T cell like cell and NK cell like cells can be in particular shown in a flow cytometric analysis (see Example 2 and FIG. 3). The cytometric analysis shows that the population of immune cells provided by the method of the invention comprises distinct sub-populations which can be characterized by the expression of distinct markers as T cell like cells and NK cell like cells. The characterization of T cell like cell and NK cell like cells will be discussed below in detail.

In a preferred embodiment of the invention, after step (ii) a further step (iii) is conducted. This step (iii) comprises inducing, selectively expanding and activating T cell like cells and NK cell like cells.

Preferably, in one embodiment of the present method T cell like cells are induced, selectively expanded and activated under suitable conditions in step (iii) Such suitable conditions for T cell like cells are, for example, expanding the cells with an CD3 activating antibody such as the antibody OKT3 antibody and/or with Interleukin 2 (IL-2). The antibody OKT3 and IL-2 activate the cells, and after the cells have been activated, they can be expanded by multiplying the cells in cell culture. The expansion of the cells may be monitored for example by determining the level of any one or more of intracellular or secreted IFN-g, TNF-a and/or IL-2.

In one preferred embodiment of the invention, in step (iii) T cell like cells are induced, selectively expanded, and activated in a medium comprising B27 supplement, SCF, Flt-3l, IL-7, IL-2, IL-15, and a CD3 stimulatory binding molecule.

In this embodiment the medium in step (iii) may comprise about 1 to about 3% B27 supplement, about 5 to about 15 ng/ml SCF, about 2 to about 7 ng/ml Flt-l3, about 5 to 15 about ng/ml IL-7, about 5 to about 15 ng/ml IL-2, about 5 to about 15 ng/ml IL-15. The CD3 stimulatory binding molecule may be selected from an anti-CD3 antibody, such as the antibody OKT-3, for example in a concentration of about 250 to about 750 ng/mL, or a multimerization reagent carrying an antigen fragment of the antibody OKT-3, for example, a Fab fragment of the antibody OKT-3. Such a multimerization reagent may be defined such that it is a multimerization reagent, wherein the multimerization reagent has reversibly immobilized thereon (bound thereto) a first agent that provides a primary activation signal to the cells; wherein the multimerization reagent comprises at least one binding site Z1 for the reversible binding of the first agent, wherein the first agent comprises at least one binding partner C1, wherein the binding partner C1 is able of reversibly binding to the binding site Z1 of the multimerization reagent, wherein the first agent is bound to the multimerization reagent via the reversible bond formed between the binding partner C1 and the binding site Z1, and wherein the first agent binds to a receptor molecule on the surface of the cells, thereby providing a primary activation signal to the cells and thereby activating the cells. Such a multimerization reagent in generally and also loaded with Fab fragments of an CD3 binding stimulatory antibody such as the antibody OKT-3 is described in detail in International Patent Application WO2015/158868. This multimerization reagent is also known under the term “Expamer”. In one illustrative embodiment of the invention, step (iii) is conducted for about 2 to about 5 days, preferably for about 3 to about 4 days.

In a preferred embodiment after step (iii) a further step (iv) is conducted comprising further expanding by culturing the immune cells under suitable conditions. In an illustrative example, this expansion may be carried out in a serum-free medium comprising B27 supplement and IL-2.

Preferably, the serum free medium of step (iv) comprises about 1% to about 3% B27 supplement and about 30 to about 70 units/mL IL-2. In a preferred embodiment of the invention, step (iv) is conducted for about 2 to 10 days, preferably for about 7 days. Preferably, step (iii) and step (iv) do not require any incubation on DLL4 coated plates, see Examples 2 and 3 of the present application.

In a preferred embodiment of the present method the population of immune cells is characterized by expression of the surface proteins CD4, CD8, CD56, CD3, and CD45. Such a characterization allows to differentiate the population of immune cells in different sub-populations of T cell like cells and NK cell like cells. For example, flow-cytometric analysis of the population of immune cells may be conducted to perform such a characterization. The person skilled in the art is able to use the marker CD4, CD8, CD56, CD3, and CD45 to define distinct sub-populations of T cell like cells and NK cell like cells. Accordingly, T cell like cells obtainable by the method of the present invention are preferably characterized by expression of CD45, CD3, either CD4 or CD8, and lack of expression of CD56, such that the T cell like cells are preferably CD45⁺, CD3⁺, CD56⁺, and CD4⁺; or CD45⁺, CD3⁺, CD56⁻, and CD8⁺. NK cell like cells obtainable by the method of the present invention are preferably characterized by expression of CD45 and CD56, and lack of expression of CD3, such that the NK cell like cells are preferably CD45⁺, CD56⁺, and CD3⁻. Other suitable markers and/or methods which allow such a distinction may be used as well Such cell selection based on the marker expression profile can be easily carried out by the person skilled in the art.

The method of the present invention is suitable of producing a population of immune cells with high cell numbers by using bioreactor conditions in any one or all of the steps (ii), (iii), and/or (iv). The use of a bioreactor provides the advantage that higher yields of cells can be achieved due to the use of a large-scale bioreactor format. The use of a bioreactor allows optimization of the concentration of different factors and at the same time production of higher cell numbers. Therefore, the number of cells of the population of immune cells provided by the method of the invention can be scaled up due to the use of a bioreactor. So, doing allows providing the number of cells of the population of immune cells need for clinical applications. In addition, so doing allows the provision of a highly standardized and cost-effective method. In addition, the use of a bioreactor simplifies the procedure, as the use of a bioreactor provides a less laborious method which can be easily conducted.

A second aspect r of the invention is directed to a population of immune cells obtainable by the method of the invention.

A third aspect of the invention is directed to a population of immune cells obtained by the method of the invention.

A fourth aspect of the invention is directed to a non-immunogenic T cell obtainable by the method of the invention.

A fifth aspect of the invention is directed to a non-immunogenic T cell obtained by the method of the invention.

A sixth aspect of the invention is directed to a non-immunogenic T cell obtainable by the method of the invention, wherein the T cell is deficient of endogenous MHC class I molecules presented on the cell surface of the T cell and the T cell comprises an immunomodulatory protein on its surface.

Preferably, the non-immunogenic T cell expresses a chimeric antigen receptor (CAR) or an exogenous T cell receptor (TCR) on its surface. A CAR protein may comprise a single-chain variable fragment (scFv) of an antibody with binding capacity for a specific tumor associated antigen, linked via a transmembrane peptide to intracellular co-stimulatory domains such as CD28, OX40 and CD137. These peptides are subsequently fused to the signaling domains of the TCRζ chain that activates the CAR T cell, if it binds its epitope on a tumor cell. Subsequent release of granzymes and perforins leads to tumor cell lysis (June C H, O'Connor R S, Kawalekar O U, Ghassemi S, Milone M C. CAR T cell immunotherapy for human cancer. Science (New York, N.Y.) 2018; 359:1361-65). These synthetic receptor molecules enable an MHC-independent T cell activation unlike the common reaction via the TCR complex. (Gideon Gross, Tova Waks, and Zelig Eshhar. Expression of immunoglobulin-T-cell receptor chimeric molecules as functional receptors with antibody-type specificity. Proc. Natl. Acad. Sci. USA 1989:10024-28; Kuwana Yea. Expression of chimeric receptor composed of immunoglobulin-derived V regions and T-cell receptor-derived C regions. Biochemical and biophysical research communications 1987:960-68). This represents an important benefit in tumor cell recognition since the loss of MHC-associated antigen presentation is a major immune escape strategy by malignant cells (Garrido F, Aptsiauri N, Doorduijn E M, Garcia Lora A M, van Hall T. The urgent need to recover MHC class I in cancers for effective immunotherapy. Current opinion in immunology 2016; 39:44-51).

In a further preferred embodiment of the invention, essentially all copies of the beta-microglobulin 2 gene are disrupted in the T cell, thereby rendering the T cell deficient of endogenous MHC class I molecules on the cell surface. In the T cell of the invention the B2M gene may be disrupted so that no functional endogenous B2M protein is produced from the disrupted genetic loci. In certain embodiments, the disruption results in expression of non-functional B2M proteins, including but not limited to truncations, deletions, point mutations and insertions. In other embodiments, the disruption results in no protein expression from the B2M gene.

Preferably, the immunomodulatory protein is a single chain fusion HLA class I protein.

In a further preferred embodiment, the single chain fusion HLA class I protein may comprise at least a portion of B2M covalently linked to at least a portion of an HLA class la chain selected from the group consisting of HLA-A, HLA-B, HLA-C, HLA-E, HLA-F and HLA-G.

Preferably, the single chain fusion HLA class I protein comprises at least a portion of B2M and at least a portion of HLA-A.

In a further preferred embodiment, the single chain fusion HLA class I protein comprises at least a portion of B2M and at least a portion of HLA-A0201.

Preferably, the single chain fusion HLA class I protein comprises at least a portion of B2M and at least a portion of HLA-E.

In a preferred embodiment the single chain fusion HLA class I protein comprises at least a portion of B2M and at least a portion of HLA-G.

Preferably, the single chain fusion HLA class I protein comprises at least a portion of B2M and at least a portion of HLA-B.

In a preferred embodiment the single chain fusion HLA class I protein comprises at least a portion of B2M and at least a portion of HLA-C.

Preferably, the single chain fusion HLA class I protein comprises at least a portion of B2M and at least a portion of HLA-F.

In a preferred embodiment the non-immunogenic T cell further expresses a target peptide antigen that is presented by the single chain fusion HLA class I protein on the pluripotent cell surface.

Preferably, the target peptide antigen is covalently linked to the single chain fusion HLA class I protein.

In a preferred embodiment the target peptide antigen comprises the sequence VMAPRTLFL (SEQ ID NO: 1).

Preferably, the gene of the immunomodulatory protein is integrated into the (endogenous) B2M locus of the T cell genome.

In a preferred embodiment the CAR or the exogenous TCR is integrated into an endogenous TCR-α gene, into an endogenous TCR-β gene or both into an endogenous TCR-α gene and an endogenous TCR-β gene.

Preferably, the CAR or the exogenous TCR binds an antigen presented on the surface of a target cell.

In a preferred embodiment the antigen is a tumor-associated antigen, a viral antigen or a bacterial antigen, preferably a tumor-associated antigen.

Preferably, the antigen is CD19 or a CMV-derived antigen.

In a preferred embodiment the T cell is a T cell of primate origin, preferably a human T cell.

A further aspect of the invention is directed to a pharmaceutical composition comprising the population of immune cells of the method of the invention, or the non-immunogenic T cell of the method of the invention.

A further aspect of the invention is directed to the population of immune cells of the invention, the non-immunogenic T cell of the invention, or the pharmaceutical composition of the invention for use in a method of treatment.

As used herein, the term “treat”, “treating” or “treatment” means to reduce (slow down (lessen)), stabilize or inhibit or at least partially alleviate or abrogate the progression of the symptoms associated with the respective disease. Thus, it includes the administration of said cell of the immune system, preferably in the form of a medicament or a pharmaceutical composition, to a subject, defined elsewhere herein. Those in need of treatment include those already suffering from the disease, here cancer. Preferably, a treatment reduces (slows down (lessens)), stabilizes, or inhibits or at least partially alleviates or abrogates progression of a symptom that is associated with the presence and/or progression of a disease or pathological condition. “Treat”, “treating”, or “treatment” refers thus to a therapeutic treatment. In particular, in the context of the present invention, treating or treatment refers to an improvement of the symptom that is associated with cancer, as defined elsewhere herein. The term “subject” when used herein includes mammalian subjects. Preferably the subject of the present invention is a mammal, including human. In some embodiment the mammal is a mouse. A subject also includes human and veterinary patients. Where the subject is a living human who may receive treatment for a disease or condition as described herein, it is also addressed as a “patient”. Those in need of treatment include those already suffering from the disease.

A further aspect of the invention is directed to the population of immune cells of the invention, for example the (non-immunogenic) T cell population or NK cell population of the invention, or the pharmaceutical composition of the invention for use in cell (based) therapy. This medical use includes administering a population of immune cells of the invention or a cell population derived from a population of immune cells of the invention to a subject in need thereof. The subject is typically a mammal such as a human, monkey, a cat, a rodent (mouse, rat), dog or another animal such as horses or cattle.

Typically, the invention is directed to the population of immune cells or the non-immunogenic T cell population or NK cell population for use according to the invention, wherein the treatment or therapy is for cancer. This means, the population of immune cells is typically to a subject for cancer treatment or immunotherapy. An immune cell population such as T cells or NK cells of the invention may be used in adoptive T cell transfer or similar therapeutic approaches. Among the different immune cell therapies such as T cell immunotherapies, adoptive cell therapy has attracted substantial attention and interest during the last years. The adoptive cell therapy is a personalized therapy in which a patient's own immune cells are removed from, expanded in vitro to large numbers, and reinfused back into the patient to eliminate tumors. A summary of the developments of the adoptive cell therapy is given in Guedan et al., Rev Immunol. 2019 Apr. 26; 37:145-171. doi: 10.1146/annurev-immunol-042718-041407.

A population of immune cells such as a (non-immunogenic) T cell population can be used for treatment of essentially all cancers. Examples of cancers that can be treated with a cell population as obtained by the invention are lung cancer, prostate cancer, ovarian cancer, testicular cancer, brain cancer, skin cancer, melanoma, colon cancer, rectal cancer, gastric cancer, esophageal cancer, tracheal cancer, head & neck cancer, pancreatic cancer, liver cancer, breast cancer, ovarian cancer, lymphoid cancers including lymphoma and multiple myeloma, leukemia, sarcomas of bone or soft tissue, cervical cancer, or vulvar cancer.

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, “about” as used herein means within 20%, preferably within 10%, and more preferably within 5% of a given value or range. It includes, however, also the concrete number, e.g., about 20 includes 20. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES OF THE INVENTION

The following examples illustrate the invention. These examples should not be construed as to limit the scope of this invention. The examples are included for purposes of illustration and the present invention is limited only by the claims.

### Example 1: Producing of a Population of Immune Cells From Pluripotent Stem Cells (PSC)

Human induced pluripotent stem (iPS) cell line TC-1133 that was reprogrammed under c-GMP conditions was purchased from Lonza. Human iPS cells were maintained on matrigel-coated T-flasks in iPS expansion medium (Miltenyi StemMACS iPS-Brew XF). Mouse stromal cells have been shown to support immune cell differentiation. To be used in the differentiation protocol, the inventors generated mouse stromal cell line that expresses Notch-ligand Delta-like ligand 4 (DLL4). Bone-marrow-derived mouse stromal cell line (MS5) was purchased from German Collection of Microorganisms and Cell Cultures GmbH. Plasmid carrying CAG promoter, full lenght human DLL4 and puromycin resistance genes and bovine growth hormone polyadenylation signal sequences in between the left and right piggybac transposase specific sequence arms was constructed. Cells were transfected with Piggybac transposase and the DLL4-carrying plasmid. Succesful transfectants were selected in puromycin containing medium and expanded. MS5-DLL4 cells were maintained in medium composed of DMEM (Gibco 42430025), 10% FBS, 1% penicillin/streptomycin, 1× Glutamax (Gibco 35050061) and 1 mM Sodium pyruvate (Gibco 11360039).

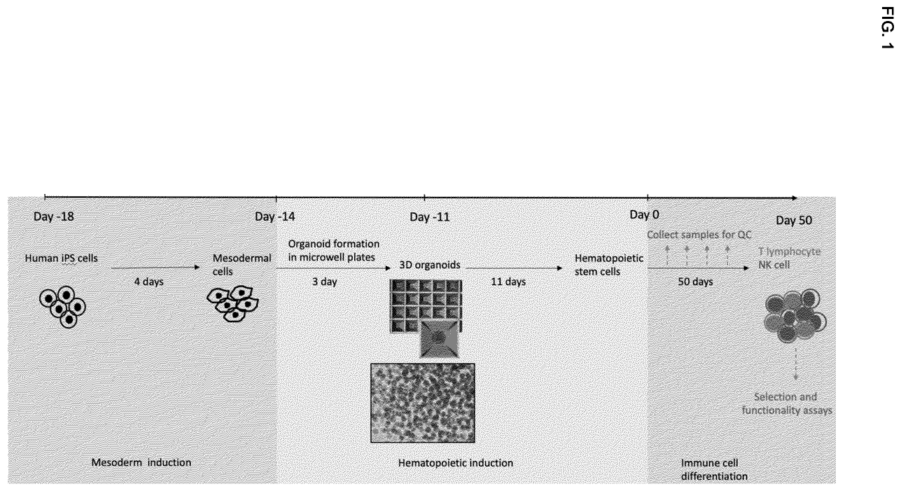

Mesoderm induction (Differentiation day −18 to day −14, see also FIG. 1 in this respect). The following steps have been carried out:
1. Human iPS cells are washed with 5 mL Dulbecco's phosphate-buffered saline (DPBS; Gibco 14190136).
2. 3 mL accutase (Gibco A1110501) are been added and the cells have been incubated in incubator at 37° C. for 4 min.
3. The cells are detached by tapping the flask and later the cells have been washed down with 5 mL DPBS.
4. The cells are transferred into falcon and centrifuge at 300 g for 4 min.
5. The supernatant is discarded and the cells are resuspended in 1 mL StemPro34-SFM medium (Gibco 10639011) and counted.
6. 1.0×10⁶ cells are seeded per one well of matrigel-coated 6-well plate in mesoderm induction medium composed of StemPro34-SFM medium supplemented with Activin A (10 ng/ml), BMP4 (10 ng/ml), VEGF (10 ng/mL), FGF (10 ng/ml), and ROCK inhibitor Y-27632 dihydrochloride (10 μM).
7. The medium is changed daily on day 1, day 2 and day 3 with StemPro34-SFM medium supplemented with BMP4 (10 ng/ml), VEGF (10 ng/ml), FGF (10 ng/ml).
8. On day 4 the cells are n washed 3 times with DPBS.
9. 1 mL accutase are added onto cells and are incubated in incubator at 37° C. for 10 min.
10. The cells are gently detached from the plate by pipetting up and down and transferred into falcon tubes.
11. A centrifugation step is conducted at 300 g for 4 min.
12. The cells are resuspended in StemPro34-SFM medium supplemented with Y-27632 and counted.

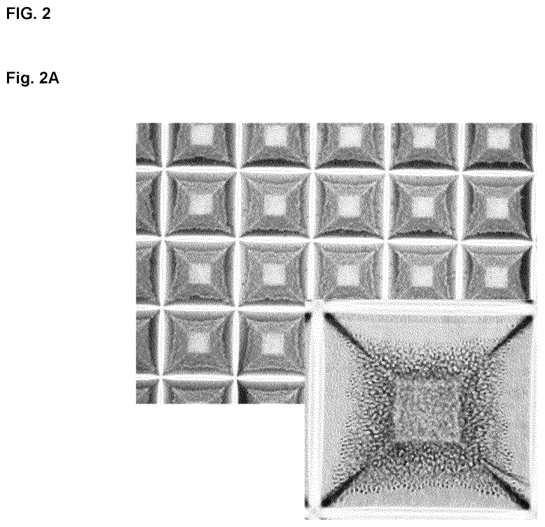

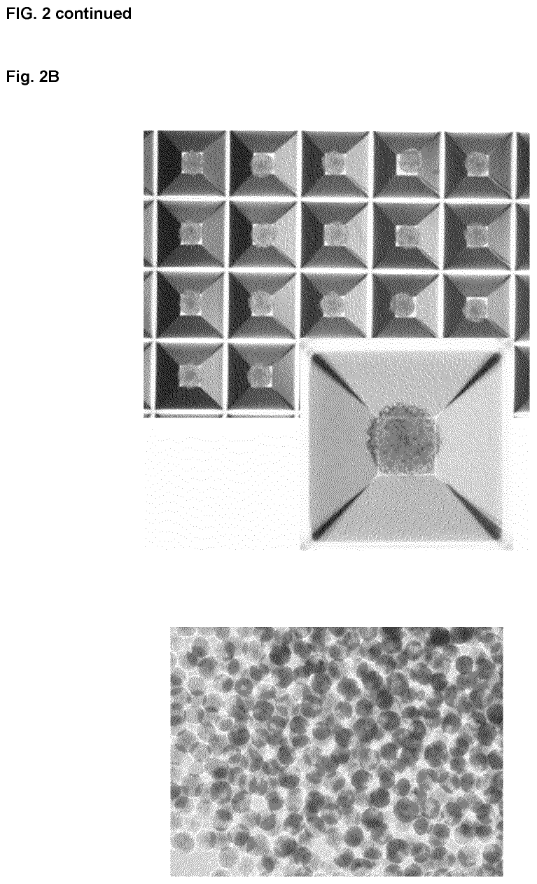

3D-aggregate formation and hematopoietic induction (Differentiation day −14 to day 0, cf. again FIG. 1). The following steps have been conducted:
1. AggreWell™ 800 6-well plate (Stemcell Technologies, 34821) are pre-treated with 2 mL Anti-Adherence Rinsing Solution (Stemcell Technologies, 07010) per well.
2. Anti-Adherence Rinsing Solution is aspirated from the wells.
3. Each well is rinsed with warm DPBS and kept at room temperature until use.
4. The MS5-DLL4 cells are harvested by trypsinization (TrypL Express, Gibco 12604013) and have been resuspended in StemPro34-SFM medium (Lonza CC-4176) and counted.
5. 5.0×10⁶ MS5-DLL4 cells are resuspended with 0.5×10⁶ mesodermal cells obtained at the end of mesoderm induction stage (per well 1:10 ratio) in 5 mL hematopoietic induction medium composed of StemPro34-SFM medium supplemented with 10 μm SB431542 and 10 μm Y-27632. The cells are gently pipeted up and down to achieve a homogeneous distribution.
6. DPBS is aspirated from the wells of the AggreWell™800 6-well plate.
7. The cells are seeded into wells carefully avoiding bubble formation.
8. The plate is r centrifugated at 100 g for 3 minutes to capture the cells in the microwells (see FIG. 2A).
9. The plate is incubated in an incubator at 37° C. with 5% CO₂ and 95% humidity for 3 days.
10. On day 3 the aggregate formation could be observed on the microscope (FIG. 2B).
11. To collect the 3D-aggregates from the plate, the plate are gently tapped and the aggregates have been dislodged. By swirling the aggregates are collected in the center of the well. Using a serological pipette, the aggregates are collected from the plate and transferred into falcon tubes.
12. After all the aggregates have settled/sunk down at the bottom of the falcon, the medium is carefully discarded without disturbing the aggregates.
13. The aggregates are resuspended in 2.5 mL hematopoietic induction medium composed of StemPro34-SFM medium supplemented with 10 μm SB431542 and are transferred into ultra-low attachment suspension culture plate.
14. The plate is placed onto an orbital shaker and maintains the aggregates in dynamic suspension culture by rotating the shaker at 80 rpm.
15. The medium is replaced every 2-3 days from differentiation day −13 to differentiation day −7 with fresh StemPro34-SFM medium supplemented with 10 μm SB431542.
16. From differentiation day −7 until differentiation day 0 the aggregates are maintained in StemPro34-SFM medium supplemented with 10 um SB431542, 50 ng/ml SCF, 5 ng/ml Flt-3l and 5 ng/ml TPO by refreshing the medium every 2-3 days.

Immune cell differentiation (Differentiation day 0 to day 50). The following step has been conducted:

From differentiation day 0 until differentiation day 50 the aggregates are maintained in dynamic suspension culture in immune cell differentiation medium composed of RPMI 1640 (Gibco 618736), 2% B27 (Gibco, 17504044), 10 ng/mL SCF, 5 ng/ml Flt-3l, 5 ng/ml IL-7, 30 mM L-ascorbic c acid 2-phosphate sesquimagnesium salt hydrate and 1% penicillin/streptomycin.

### Example 2: Activation and Flow Cytometric Analyses of the Differentiated Cells

The following steps have been conducted:
1. On differentiation day 50 the aggregates are collected from the culture plates and have been washed once with DPBS.
2. The aggregates are dissociated into single cells in dissocation solution composed of 1 mg/mL collagenase type, 1 mg/mL collagenase/dispase and 5000 units/mL for 1 hour at 37° C.
3. The dissociated cells are centrifuged and resuspended in activation medium composed of RPMI 1640 (Gibco 618736), 2% B27 (Gibco, 17504044) and 50 units/mL IL-2.
4. The cells are seeded into cell culture flask and culture in an incubator at 37° C. with 5% CO₂ and 95% humidity for one week.
5. One week later, the cells are collected from the cell culture flask and passaged through 100 μm sterile filters.
6. The singularized cells are pelleted by centrifugation, washed once with PBS and resuspended in staining buffer composed of PBS and 0.5% human serum albumin.
7. The cells are stained with anti-mouse anti-CD29 and anti-human anti-CD45, anti-CD3, anti-CD4, anti-CD8, anti-CD19 and anti-CD56 for 20 min in 4° C. in the presence of Fc-block.
8. The cells arecwashed with PBS and resuspended in staining buffer containing live/dead dye (PI).
9. The cells are analyzed on a Cytoflex LX flow cytometer according to the manufacturer's instructions.
   The results of this experiments are shown in FIG. 3. The following gating strategy was applied. Cell progenitors were pre-gated on single, live, lymphocyte-like cell population according to side and forward scatter. To separate progenitors from feeders, cells were gated according to CD29/CD45 features. All CD29−CD45+ cells were characterized for phenotype of CD3+CD56− (T cell lineage marker), CD3−CD56+CD8+/− (NK cell lineage) and CD19+ (B cell lineage). Moreover, CD3+CD56− T cell like cells were further characterized for expression of CD4 and CD8 markers. These cells exhibited expression pattern similar to classical thymocyte profile of CD4−CD8−, CD4+CD8+, CD4−CD8+and CD4+CD8−.

### Example 3: Earlier Maturation of the Cells of the Population of Immune Cells With Anti-CD3 Antibody Stimulation

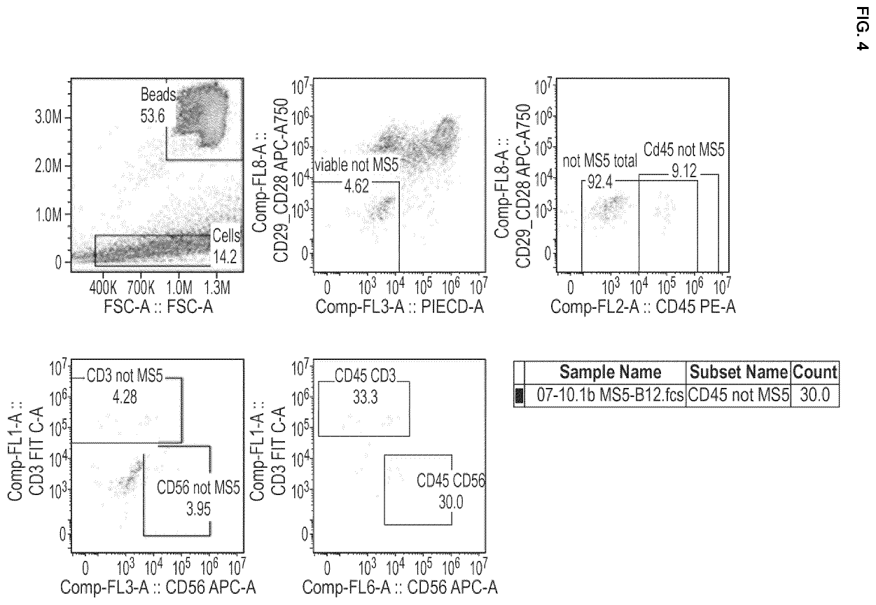

In order to achieve the earlier maturation of the cells of the population of immune cells, the following steps have been conducted:
1. On differentiation week 3 (of immune cell differentiation stage) the aggregates are collected from the culture plates and have been washed once with DPBS.
2. The aggregates are dissociated into single cells in dissociation solution composed of 1 mg/ml collagenase type II.
3. The dissociated cells are centrifuged and resuspended in maturation medium composed of RPMI 1640 (Gibco 618736), 2% B27 (Gibco, 17504044), 10 ng/ml SCF, 5 ng/ml Flt-3l and 10 ng/ml IL-7, 10 ng/ml IL-2, 10 ng/ml IL-15 and 500 ng/ml monoclonal anti-human anti-CD3 antibody (Biolegend 317347).
4. The cells are seeded into cell culture flask and cultured at 37° C. in an incubator with 5% CO₂ and 95% humidity for one week.
5. One week later, the cells are collected from the cell culture flask and are passaged through 100 μm sterile filters.
6. The singularized cells are pelleted by centrifugation, washed once with PBS and resuspended in staining buffer composed of PBS and 0.5% human serum albumin.
7. The cells are stained with anti-mouse anti-CD29 and anti-human anti-CD45, anti-CD3 and anti-CD56 for 20 min in 4° C. in the presence of Fc-block.
8. The cells are washed with PBS and resuspended in staining buffer containing live/dead dye (PI).
9. The cells are analyzed on Cytoflex LX flow cytometer according to the manufacturer's instructions.
   The results are shown in FIG. 4. The following gating strategy was. Cell progenitors were pre-gated on single, live, lymphocyte-like cell population according to side and forward scatter. To separate progenitors from feeders, cells were gated according to CD29/CD45 features. All CD29−CD45+ cells were characterized for phenotype of CD3+CD56− (T cell lineage marker), CD3−CD56+ (NK cell lineage).

### Example 4: Differentiation Into NK Like Cells

The following steps have been conducted:
1. On differentiation day 50 the aggregates are collected from the culture plates and washed once with DPBS.
2. The aggregates are dissociated into single cells in dissocation solution composed of 1 mg/mL collagenase type, 1 mg/mL collagenase/dispase and 5000 units/mL for 1 hour at 37° C.
3. The dissociated cells are analysed and selected for CD45+ CD56+ NK cell like cells.
4. These CD45+ CD56+ NK cell like cells are centrifuged and resuspended in medium composed of RPMI 1640 (Gibco 618736), 2% B27 (Gibco, 17504044) and 50 units/mL IL-2 and IL-15.
5. The cells are seeded into cell culture flask and culture in an incubator at 37° C. with 5% CO2 and 95% humidity for 13 days.

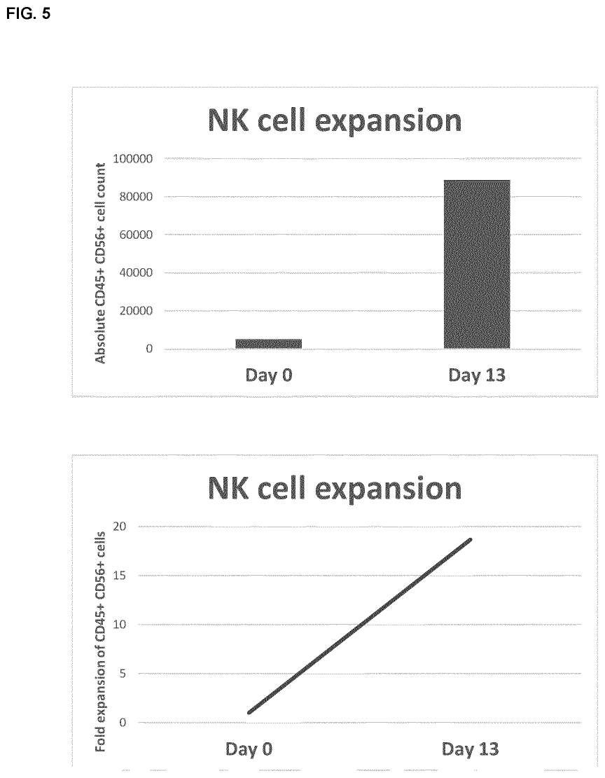

The results are shown in FIG. 5. It was found hat the CD45+ CD56+ NK cell like cells have been further expanded 19-fold after 13 days of IL-2 and IL-15 cytokine stimulation.

### Example 5: Measuring of the Cytotoxic Activity of the Differentiated Immune Cells

Cytoxic activity of the differentiated immune cells has been measured using a lactate dehydrogenase (LDH) activity assay, in the following LDH assay. Lactate dehydrogenase (LDH) is an oxidoreductase enzyme that catalyses the interconversion of pyruvate and lactate. Cells release LDH after tissue damage. Since LDH is a quite stable enzyme, it is useful to evaluate the presence of damage and toxicity of tissue and cells. In the LDH assay as described in the following K562 cells, which are the target cells within the assay, have been cocultured with the differentiated immune cells of the invention with the following target effector ratios of 1:1, 1:2, 1:4 and 1:8.

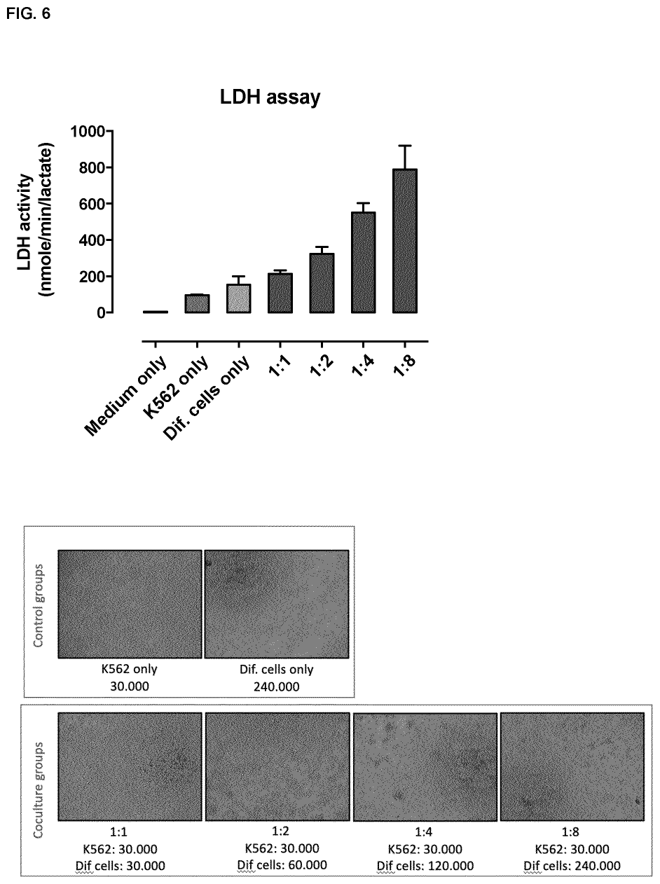

The following steps have been conducted:
1. The differentiated immune cells are cultured for three days in advance of conducting the LDH assay in immune cell differentiation medium with IL-2, said medium comprises the following:
   Iscove's Modified Dulbecco's Medium (IMDM) (thermo fischer sci Cat No 31980030),
   15% BIT 9500 serum substitute (stem cell tech Catalog #09500),
   1% penicillin streptomycin,
   L-ascorbic acid 2-phophatate sesquimagnesium salt hydrate (30 mM) (the range can be from
   15 mM to 50 mM),
   SCF (10 ng/ml) (the range can be from 5 ng/ml to 50 ng/ml),
   TPO (10 ng/ml) (the range can be from 5 ng/ml to 50 ng/mL),
   Flt3l (10 ng/mL) (the range can be from 5 ng/ml to 50 ng/mL),
   IL-7 (10 ng/ml) (the range can be from 5 ng/ml to 50 ng/ml),
   IL-15 (10 ng/mL) (the range can be from 5 ng/ml to 50 ng/ml),
   IL-2 (10 ng/ml) (the range can be from 5 ng/ml to 20 ng/ml).
2. K562 cells have been prepared by seeding the K562 cells in a cell culture well-plate such that 3000 cells per well are added. As a control, wells including K562 cells only have been prepared as well—accordingly said control wells are not for coculture with differentiated immune cells.
3. Differentiated immune cells were added with the respective ratios of 1:1, 1:2, 1:4 and 1:8. As a further control, distinct wells included differentiated immune cells only—not in coculture with K562 cells—with equal numbers of differentiated immune cells such as in the wells of the 1:8 ratio.
4. The cocultures and controls were cultured in immune cell differentiation medium with IL-2 for 24 hours at 37° C. in 5% CO₂.
5. After 24 hours, the medium was collected and the LDH assay was conducted and measured according to the manufacturer's protocol (Sigma Aldrich, catalog number MAK066-1KT). The percentage of specific release of LDH was calculated. The data are shown in FIG. 6. The data shown therein represent the means +/−SD of triplicate cultures. The result of the LDH assay could show that the cellular cytoxicity measured by LDH activity was significantly higher in the 1:4 and 1:8 ratio groups of coculture samples as compared to the control groups. Further, a microscopic analyses has been conducted, also shown in FIG. 6. According to the microscope analyses, K562 cells were observed to be very low or absent in the 1:4 and 1:8 mixed cocultures.

In summary, cytotoxic activity could be observed for the differentiated immune cells of the invention.

1. A method of producing a population of immune cells from pluripotent stem cells (PSC), the method comprising the steps of:
(i) inducing 3D cell aggregate formation and hematopoietic differentiation by
(a) culturing under suitable conditions PSC having undergone mesenchymal differentiation in the presence of Notch-ligand Delta-like ligand 4 (DLL4) signaling activity, on a solid support suitable for formation of 3D-cell aggregates, in a serum-free medium, thereby allowing the formation of 3D-cell aggregates;
(b) collecting the 3D-cell aggregates and culturing under suitable conditions in a suspension culture in a serum-free medium for about 3 to 6 days; and
(c) culturing the 3D-cell aggregates under suitable conditions for about further 4 to 7 days in a serum-free medium;
(ii) inducing immune cell differentiation by culturing the 3D-cell aggregates of (i) under suitable conditions in a suspension culture for a suitable time in a serum-free medium;
thereby providing a population of immune cells.

2. The method of claim 1, wherein before step (i) a further step (0i) is conducted, which comprises:
(0i) inducing mesodermal differentiation by
(a) seeding PSC under suitable conditions in a serum-free medium; and
(b) culturing the PSC under suitable conditions in a serum-free medium for about 2 to 4 days, and
(c) resuspend cells with serum-free medium.

3. The method of claim 1 or 2, wherein the serum-free medium comprises:
in step (0i) (a) Activin A, bone morphogenic protein 4 (BMP4), vascular endothelial growth factor (VEGF), fibroblast growth factor (FGF), and an apoptosis inhibitor, preferably a ROCK inhibitor, each in a concentration of about 10 to 50 ng/mL;
(b) BMP4, VEGF, and FGF, each in a concentration of about 5 to 15 ng/ml;
(c) an apoptosis inhibitor, preferably a ROCK inhibitor;
in step (i) (a) an inhibitor of ALK receptors and a ROCK inhibitor, each in a concentration of about 5 to 15 μM,
(b) about 5 to 15 ng/ml of an inhibitor of ALK receptors,
(c) about 5 to 15 ng/ml of an inhibitor of ALK receptors, about 40 to 60 ng/ml stem cell factor (SCF), about 5 to 15 ng/mL FMS-like tyrosine kinase 3 ligand (Flt-3l), and about 5 to 15 ng/mL Thrombopoietin (TPO);
in step (ii) about 1 to 3% B27 supplement, about 5 to 15 ng/ml SCF, about 5 to 15 ng/ml Flt-3l, about 5 to 15 ng/ML IL-7, and about 15 to 45 mM L-ascorbic acid 2-phophatate sesquimagnesium salt hydrate.

4. The method of any one of the preceding claims, wherein the ROCK inhibitor is Y-27632 dihydrochloride and the inhibitor of ALK receptors is SB43152.

5. The method of any one of the preceding claims, wherein the suspension culture in step (i) and (ii) is a dynamic suspension culture, preferably by culturing on a shaker, such as an orbital shaker, at rotations of about 60 to 80 rpm.

6. The method of any one of the preceding claims, wherein the step (ii) is conducted for about 21 to 50 days.

7. The method of any one of the preceding claims, wherein the PSC are human induced pluripotent stem (iPS) cells, preferably the human iPS cell line TC-1133.

8. The method of any one of the preceding claims, wherein the PSC are pluripotent stem cells being deficient of endogenous MHC class I molecules presented on the cell surface of the pluripotent stem cell and comprising an immunomodulatory protein on their surface

9. The method of claim 8, wherein the immunomodulatory protein is a single chain fusion HLA class I protein.

10. The method of claim 8 or 9, wherein the single chain fusion HLA class I protein comprises at least a portion of B2M covalently linked to at least a portion of an HLA class 1α chain selected from the group consisting of HLA-A, HLA-B, HLA-C, HLA-E, HLA-F and HLA-G.

11. The method of any one of claims 8 to 10, wherein the pluripotent stem cell further expresses a target peptide antigen that is presented by the single chain fusion HLA class I protein on the pluripotent cell surface.

12. The method of claim 11, wherein the target peptide antigen is covalently linked to the single chain fusion HLA class I protein.

13. The method of claim 11 or 12, wherein the target peptide antigen comprises the sequence VMAPRTLFL (SEQ ID NO: 1).

14. The method of any one of claims 8 to 13, wherein essentially all copies of the beta-microglobulin 2 gene are disrupted in the pluripotent stem cells.

15. The method of any one of claims 8 to 14, wherein the pluripotent stem cells are selected from the group consisting of embryonic stem cells, induced pluripotent stem cells and parthenogenetic stem cells.

16. The method of any one of claims 8 to 15, wherein the pluripotent stem cells are pluripotent stem cells of primate origin, preferably human pluripotent stem cells.

17. The method of any one of claims 8 to 16, wherein the pluripotent stem cell is generated from CD34-positive cell isolated form umbilical cord blood.

18. The method of any one of claims 8 to 17, wherein the pluripotent stem cell is ND-50039 of the NINDS Human Cell and Data Repository.

19. The method according to any one of the claims 1 to 7, wherein the PSC are pluripotent stem cells being deficient of endogenous MHC class II.

20. The method according to claim 19, wherein the PSC are deficient of endogenous MHC class Il by disrupting the C2TA gene-MHC class II transactivator.

21. The method of any one of the preceding claims, wherein in step (i) Notch-ligand Delta-like ligand 4 (DLL4) signaling activity is provided by co-culturing PSC with stromal cells expressing DLL4, or by incubating PSC with beads which are coated with DLL4.

22. The method of any one of the preceding claims, wherein the ratio of PSC to stromal cells is in the range of about 1:5 to 1:40.

23. The method of any one of the preceding claims, wherein in step (i) the stromal cells are the bone marrow-derived mouse stromal cell line MS5.

24. The method of any one of the preceding claims, wherein in step (i) the stromal cells are stromal cells differentiated from PSC expressing DLL4.

25. The method of any one of the preceding claims, wherein the PSC during induction of mesodermal differentiation in step (0i) are cultured in a cell culture container comprising at least one extracellular matrix protein.

26. The method of claim 25, wherein the container is coated with the at least one extracellular matrix protein.

27. The method of claim 25 or claim 26, wherein the at least one extracellular matrix protein is selected from the group consisting of vitronectin, laminin, collagen, fibronectin, elastin, Matrigel, a peptide containing the amino acid sequence RGD, a protein containing the amino acid sequence RGD and combinations thereof.

28. The method of any one of the preceding claims, wherein in step (i)(a) the solid support comprises one or more wells, wherein each well comprises a V-shaped or conical cavity.

29. The method of claim 28, wherein the solid support is a microwell culture plate.

30. The method of claim 29, wherein the microwell culture plate is an AggreWell™ plate (available from StemCell Technologies), an BIOFLOAT™ 96-well plate (available from faCellitate), or an Nunclon Sphera 3D culture (available from ThermoFisher).

31. The method of any one of the preceding claims, wherein the 3D-cell aggregates after step (ii) are separated such that single cells are provided.

32. The method of any one of the preceding claims, wherein the population of immune cells comprises T cell like cells and NK cell like cells.

33. The method of any one of the preceding claims, wherein after step (ii) a further step (iii) is conducted comprising inducing, and preferably also selectively expanding, and activating T cell like cells and NK cell like cells.

34. The method of any one of the preceding claims, wherein in step (iii) T cell like cells are induced, selectively expanded, and activated under suitable conditions.

35. The method of any one of the preceding claims, wherein in step (iii) T cell like cells are selectively expanded in a medium comprising B27 supplement, SCF, Flt-3l, IL-7, IL-2, IL-15, and a CD3 stimulatory binding molecule.

36. The method of claim 35, wherein the medium in step (iii) comprises about 1 to 3% B27 supplement, about 5 to 15 ng/ml SCF, about 2 to 7 ng/ml Flt-l3, about 5 to 15 ng/ml IL-7, about 5 to 15 ng/ml IL-2, about 5 to 15 ng/ml IL-15, and wherein the CD3 stimulatory binding molecule is selected from an anti-CD3 antibody, such as an OKT-3 antibody, preferably in a concentration of about 250 to 750 ng/ml, and an multimerization reagent carrying an OKT-3 Fab fragment.

37. The method of any one of the preceding claims, wherein after step (iii) a further step (iv) is conducted comprising further expanding by culturing the immune cells under suitable conditions in a serum-free medium comprising B27 supplement and IL-2.

38. The method of any one of the preceding claims, wherein the serum free medium of step (iv) comprises 1 to 3% B27 supplement and 30 to 70 units/mL IL-2.

39. The method of any one of the preceding claims, wherein the population of immune cells is characterized by expression of CD4, CD8, CD56, CD3, and CD45.

40. The method of any one of the preceding claims, wherein the method is suitable for production of a population of immune cells with high cell numbers by using bioreactor conditions in any one or all of the steps (ii), (iii), and/or (iv).

41. A population of immune cells obtainable by the method of any one of claims 1 to 40.

42. A population of immune cells obtained by the method of any one of claims 1 to 40.

43. A non-immunogenic T cell obtainable by the method of any one of claims 1 to 40.

44. A non-immunogenic T cell obtained by the method of any one of claims 1 to 40.

45. A non-immunogenic T cell obtainable by the method of any one of claims 1 to 40, wherein the T cell is deficient of endogenous MHC class I molecules presented on the cell surface of the T cell and the T cell comprises an immunomodulatory protein on its surface.

46. The non-immunogenic T cell of claim 45, wherein the non-immunogenic T cell expresses a chimeric antigen receptor (CAR) or an exogenous T cell receptor (TCR) on its surface.

47. The non-immunogenic T cell of claim 45 or 46, wherein essentially all copies of the beta-microglobulin 2 gene are disrupted in the T cell, thereby rendering the T cell deficient of endogenous MHC class I molecules on the cell surface.

48. The non-immunogenic T cell of any one of claims 45 to 47, wherein the immunomodulatory protein is a single chain fusion HLA class I protein.

49. The non-immunogenic T cell of claim 48, wherein the single chain fusion HLA class I protein comprises at least a portion of B2M covalently linked to at least a portion of an HLA class Iα chain selected from the group consisting of HLA-A, HLA-B, HLA-C, HLA-E, HLA-F and HLA-G.

50. The non-immunogenic T cell of claim 48 or 49 wherein the single chain fusion HLA class I protein comprises at least a portion of B2M and at least a portion of HLA-A.

51. The non-immunogenic T cell of any one of claims 45 to 50, wherein the single chain fusion HLA class I protein comprises at least a portion of B2M and at least a portion of HLA-A0201.

52. The non-immunogenic T cell of claim 48 or 49, wherein the single chain fusion HLA class I protein comprises at least a portion of B2M and at least a portion of HLA-E.

53. The non-immunogenic T cell of claim 48 or 49, wherein the single chain fusion HLA class I protein comprises at least a portion of B2M and at least a portion of HLA-G.

54. The non-immunogenic T cell of claim 48 or 49, wherein the single chain fusion HLA class I protein comprises at least a portion of B2M and at least a portion of HLA-B.

55. The non-immunogenic T cell of claim 48 or 49, wherein the single chain fusion HLA class I protein comprises at least a portion of B2M and at least a portion of HLA-C.

56. The non-immunogenic T cell of claim 48 or 49, wherein the single chain fusion HLA class I protein comprises at least a portion of B2M and at least a portion of HLA-F.

57. The non-immunogenic T cell of any one of claims 45 to 56, wherein the non-immunogenic T cell further expresses a target peptide antigen that is presented by the single chain fusion HLA class I protein on the pluripotent cell surface.

58. The non-immunogenic T cell of claim 57, wherein the target peptide antigen is covalently linked to the single chain fusion HLA class I protein.

59. The non-immunogenic T cell of claim 57 or 58, wherein the target peptide antigen comprises the sequence VMAPRTLFL (SEQ ID NO: 1).

60. The non-immunogenic T cell of any one of claims 45 to 59, wherein the gene of the immunomodulatory protein is integrated into the (endogenous) B2M locus of the T cell genome.

61. The non-immunogenic T cell of any one of claims 45 to 60, wherein the CAR or the exogenous TCR is integrated into an endogenous TCR-α gene, into an endogenous TCR-β gene or both into an endogenous TCR-α gene and an endogenous TCR-β gene.

62. The non-immunogenic T cell of any one of claims 45 to 61, wherein the CAR or the exogenous TCR binds an antigen presented on the surface of a target cell.

63. The non-immunogenic T cell of claim 62, wherein the antigen is a tumor-associated antigen, a viral antigen or a bacterial antigen, preferably a tumor-associated antigen.

64. The non-immunogenic T cell of claim 62 or 63, wherein the antigen is CD19 or a CMV-derived antigen.

65. The non-immunogenic T cell of claims 45 to 64, wherein the T cell is a T cell of primate origin, preferably a human T cell.

66. A pharmaceutical composition comprising the population of immune cells of claim 41 or 42, or the non-immunogenic T cell of any one of claims 43 to 65.

67. The population of immune cells of claim 41 or 42, the non-immunogenic T cell of any one of claims 43 to 65, or the pharmaceutical composition of claim 66 for use in a method of treatment.

68. The population of immune cells of claim 41 or 42, the non-immunogenic T cell of any one of claims 43 to 65, or the pharmaceutical composition of claim 66 for use in cell therapy.

69. The population of immune cells or the non-immunogenic T cell for use of claim 67 or 68, wherein the treatment or therapy is for cancer.

70. The population of immune cells or the non-immunogenic T cell for use of claim 69, wherein the cancer is selected from lung cancer, prostate cancer, ovarian cancer, testicular cancer, brain cancer, skin cancer, melanoma, colon cancer, rectal cancer, gastric cancer, esophageal cancer, tracheal cancer, head & neck cancer, pancreatic cancer, liver cancer, breast cancer, ovarian cancer, lymphoid cancers including lymphoma and multiple myeloma, leukemia, sarcomas of bone or soft tissue, cervical cancer, and vulvar cancer.


SEQUENCE LISTING (1 sequence)

SEQ ID NO: 1 - protein, 9 aa, synthetic construct
  REGION 1..9: note = Peptide antigen
VMAPRTLFL